(19) United States
(12) Patent Application Publication
(10) Pub. No.: US 2022/0047290 A1
(43) Pub. Date: Feb. 17, 2022

(54) SYSTEMS AND METHODS OF ENDOSCOPIC INSTRUMENTS WITH ARTICULATING END

(71) Applicant: Interscope, Inc., Whitinsville, MA (US)

(72) Inventors: Jeffery B. Ryan, Jr., Whitinsville, MA (US); Corey Libby, Whitinsville, MA (US); Stephan Evans, Whitinsville, MA (US); Nathan Casey, Whitinsville, MA (US)

(21) Appl. No.: 17/293,226

(22) PCT Filed: Nov. 13, 2019
(86) PCT No.: PCT/US2019/061295

§ 371 Date: May 12, 2021

Related U.S. Application Data
(60) Provisional application No. 62/760,344, filed on Nov. 13, 2018.

(51) Int. Cl. A61B 17/32; A61B 17/00

(52) U.S. Cl. CPC A61B 17/32002; A61B 17/00234; A61B 2017/00314; A61B 2017/00323; A61B 2017/0034; A61B 2217/007; A61B 2017/00455; A61B 2017/00199; A61B 2017/00398; A61B 2017/00867; A61B 2217/005; A61B 2017/00296

(57) ABSTRACT

An endoscopic instrument (312, 820, 824) includes an outer tubing (206) extending from a proximal tubing end to a distal tubing end, a flexible torque component (200) extending through the outer tubing, an articulation assembly (400, 500, 600, 700, 828), and an end effector (450, 550, 650, 750, 824). The articulation assembly (400, 500, 600, 700, 828) is coupled to the distal tubing end and includes a plurality of segments (404, 408, 504, 604, 704) and at least one control member (412, 512, 612, 712) extending from a proximal control end to a distal control end coupled with the plurality of segments. The control member manipulates an orientation of at least one segment of the plurality of segments responsive to receiving a control input at the proximal control end. The end effector (450, 550, 650, 750, 824) is coupled to a distal end of the flexible torque component such that manipulating the orientation (516, 616, 620) of the at least one segment controls an orientation of the end effector (450, 550, 650, 750, 824) relative to the outer tubing (206) and rotation of the flexible torque component (200) controls an angle of rotation of the end effector relative (450, 550, 650, 750, 824) to the outer tubing (206).

### CROSS-REFERENCE TO RELATED APPLICATIONS

The present disclosure claims the benefit of priority to U.S. Provisional Application No. 62/760,344, titled “SYSTEMS AND METHODS OF ENDOSCOPIC INSTRUMENTS WITH ARTICULATING END,” filed Nov. 13, 2018, the disclosure of which is incorporated herein by reference in its entirety.

### BACKGROUND

Colon cancer is the third leading cause of cancer in the United States but is the second leading cause of cancer-related deaths. Colon cancer arises from pre-existing colon polyps (adenomas) that occur in as many as 35% of the US population. Colon polyps can either be benign, precancerous or cancerous. Colonoscopy is widely regarded as an excellent screening tool for colon cancer that is increasing in incidence worldwide. According to the literature, a 1% increase in colonoscopy screening results in a 3% decrease in the incidence of colon cancer. The current demand for colonoscopy exceeds the ability of the medical system to provide adequate screening. Despite the increase in colon cancer screening the past few decades, only 55% of the eligible population is screened, falling far short of the recommended 80%, leaving millions of patients at risk.

Due to the lack of adequate resources, operators performing a colonoscopy typically only sample the largest polyps, exposing the patient to sample bias by typically leaving behind smaller less detectable polyps that could advance to colon cancer prior to future colonoscopy. Because of the sample bias, a negative result from the sampled polyps does not ensure the patient is truly cancer-free. Existing polyps removal techniques lack precision are cumbersome and time consuming.

At present, colon polyps are removed using a snare that is introduced into the patient's body via a working channel defined within an endoscope. The tip of the snare is passed around the stalk of the polyp to cut the polyp from the colon wall. Once the cut has been made, the cut polyp lies on the intestinal wall of the patient until it is retrieved by the operator as a sample. To retrieve the sample, the snare is first removed from the endoscope and a biopsy forceps or suction is fed through the same channel of the endoscope to retrieve the sample.

### SUMMARY

This Summary is provided to introduce a selection of concepts in a simplified form that are further described below in the Detailed Description. This Summary is not intended to identify key features or essential features of the claimed subject matter, nor is it intended that this Summary be used to limit the scope of the claimed subject matter. Furthermore, the claimed subject matter is not limited to implementations that offer any or all advantages or solve any or all state of the art problems.

An improved endoscopic instrument is provided that can precisely remove sessile polyps and efficiently obtain samples of multiple polyps from a patient. In particular, the improved endoscopic instrument is capable of debriding one or more polyps and retrieving the debrided polyps without having to alternate between using a separate cutting tool and a separate sample retrieving tool. The sampling can be integrated with colonoscopy inspection. In some implementations, the endoscopic instrument can cut and remove tissue from within a patient. In some such implementations, the endoscopic instrument can cut and remove tissue substantially simultaneously from within a patient accessed through a flexible endoscope. In some implementations, a distal end of the endoscopic tool can be articulated, enabling the improved endoscopic tool to more effectively remove samples from difficult-to-reach geometries in tortuous pathways.

At least one aspect relates to an endoscope system. The endoscope system can include an endoscope and an endoscopic instrument. The endoscope can include an instrument channel, an external sheath coupled to the endoscope, or both. The endoscopic instrument can be inserted through at the instrument channel or the external sheath. The endoscopic instrument can include an outer tubing extending from a proximal tubing end to a distal tubing end. The endoscopic instrument can include a flexible torque component extending through the outer tubing. The flexible torque component can include at least one of a flexible torque wire or a flexible torque coil. The articulation assembly can be coupled to the distal tubing end of the outer tubing, and can include a plurality of segments and at least one control member extending from a proximal control end to a distal control end coupled with the plurality of segments. The at least one control member can manipulate an orientation of at least one segment of the plurality of segments responsive to receiving a control input at the proximal control end. The endoscopic instrument can include an end effector coupled to a distal end of the flexible torque component such that manipulating the orientation of the at least one segment controls an orientation of the end effector relative to the longitudinal axis of the endoscope and rotation of the flexible torque component controls an angle of rotation of the end effector relative to an effector axis of the end effector. Manipulating the orientation may include manipulating a pose (e.g., position and orientation) of the end effector. As such, the endoscopic instrument can be used to articulate the end effector to a greater range of orientations and positions, even after passing through a tortuous pathway.

At least one aspect relates to an endoscopic instrument. The endoscopic instrument can include an outer tubing extending from a proximal tubing end to a distal tubing end. The endoscopic instrument can include a flexible torque component extending through the outer tubing. The flexible torque component can include at least one of a flexible torque wire or a flexible torque coil. The articulation assembly can be coupled to the distal tubing end of the outer tubing, and can include a plurality of segments and at least one control member extending from a proximal control end to a distal control end coupled with the plurality of segments. The at least one control member can manipulate an orientation of at least one segment of the plurality of segments responsive to receiving a control input at the proximal control end. The endoscopic instrument can include an end effector coupled to a distal end of the flexible torque component such that manipulating the orientation of the at least one segment controls an orientation of the end effector relative to the outer tubing and rotation of the flexible torque component controls an angle of rotation of the end effector relative to the outer tubing.

At least one aspect relates to a method for controlling an end effector of an endoscopic instrument. The method can include providing the endoscopic instrument through at least one of an instrument channel of an endoscope or an external sheath coupled to the endoscope, a distal end of the endoscope positioned in proximity to a site within a subject, determining an orientation at which to articulate the end effector to contact material at the site within the subject, providing a first control input to at least one control member of the endoscopic instrument based on the orientation to cause an articulation assembly of the endoscopic instrument to move the end effector to the orientation, and providing a second control input to a flexible torque component coupled to the end effector to cause the end effector to interact with the material responsive to the second control input.

### BRIEF DESCRIPTION OF THE DRAWINGS

The present disclosure is illustratively shown and described in reference to the accompanying drawing in which:

FIG. 1 illustrates various types of polyps that can form within a body.

FIG. 2 is an exploded perspective view of an improved endoscopic tool according to embodiments of the present disclosure.

FIG. 3 is a schematic diagram of a colon of a subject.

FIG. 4A is a perspective view of an articulation assembly of an endoscopic instrument according to embodiments of the present disclosure.

FIG. 4B is a section view the articulation assembly of FIG. 4A.

FIG. 4C is a side view of the articulation assembly of FIG. 4A in an articulated configuration.

FIG. 4D is a side view of the articulation portion of FIG. 4A coupled to a cutting assembly.

FIG. 5A is a perspective view of an articulation assembly of an endoscopic instrument according to embodiments of the present disclosure.

FIG. 5B is a side view the articulation assembly of FIG. 5A.

FIG. 5C is a side view of the articulation assembly of FIG. 5A in an articulated configuration.

FIG. 5D is a side view of the articulation assembly of FIG. 5A coupled to a cutting assembly.

FIG. 6A is a perspective view of an articulation assembly of an endoscopic instrument according to embodiments of the present disclosure.

FIG. 6B is a section view the articulation assembly of FIG. 6A.

FIG. 6C is a section view of the articulation assembly of FIG. 6A in an articulated configuration.

FIG. 6D is a side view of the articulation assembly of FIG. 6A coupled to a cutting assembly.

FIG. 7A is a perspective view of an articulation assembly of an endoscopic instrument according to embodiments of the present disclosure.

FIG. 7B is a schematic diagram of couplings of conductors and segments of the articulation assembly of FIG. 7A.

FIG. 7C is a detail view of a segment of the articulation assembly of FIG. 7A.

FIG. 7D is a detailed perspective view of a segment of the articulation assembly of FIG. 7A

FIG. 7E is a side view of the articulation assembly of FIG. 7A in an articulated configuration.

FIG. 7F is a side view of the articulation assembly of FIG. 7A coupled to a cutting assembly.

FIG. 8 is a schematic diagram of an endoscope system including an articulation assembly.

FIG. 9 is a flow diagram of a method for controlling an endoscopic instrument.

### DETAILED DESCRIPTION

Technologies provided herein are directed towards an improved flexible endoscopic instrument that can precisely and efficiently obtain samples of single and multiple polyps and neoplasms from a patient. In particular, the improved endoscopic instrument is capable of debriding samples from one or more polyps and retrieving the debrided samples without having to remove the endoscopic instrument from the treatment site within the patient's body, including from treatment sites that are accessed via tortuous pathways.

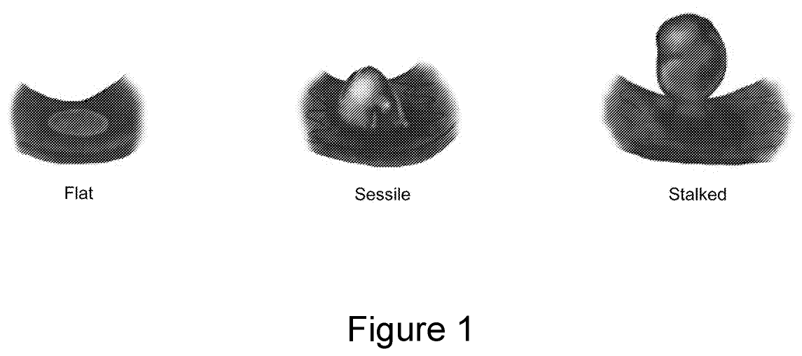

FIG. 1 illustrates various types of polyps that can form within a body. Most polyps may be removed by snare polypectomy, though especially large polyps and/or sessile or flat polyps must be removed piecemeal with biopsy forceps or en bloc using endoscopic mucosal resection (EMR). A recent study has concluded that depressed sessile polyps had the highest rate for harboring a malignancy at 33%. The same study has also found that non-polypoid neoplastic lesions (sessile polyps) accounted for 22% of the patients with polyps or 10% of all patients undergoing colonoscopy. There are multiple roadblocks to resecting colon polyps, namely the difficulties in removing sessile polyps, the time involved in removing multiple polyps and the lack of reimbursement differential for resecting more than one polyp. Since resecting less accessible sessile polyps presents challenges and multiple polyps take more time per patient, most polyps are removed piece meal with tissue left behind as polyps increase in size, contributing to a sampling bias where the pathology of remaining tissue is unknown, leading to an increase in the false negative rate.

Colonoscopy is not a perfect screening tool. With current colonoscopy practices the endoscopist exposes the patient to sample bias through removal of the largest polyps (stalked polyps), leaving behind less detectable and accessible sessile/flat polyps. Sessile polyps are extremely difficult or impossible to remove endoscopically with current techniques and often are left alone. An estimated 28% of stalked polyps and 60% of sessile (flat) polyps are not detected, biopsied or removed under current practice, which contributes to sample bias and a 6% false-negative rate for colonoscopy screening. Current colonoscopy instruments for polyp resection are limited by their inability to adequately remove sessile polyps and inefficiency to completely remove multiple polyps. According to the clinical literature, sessile polyps greater than 10 mm have a greater risk of malignancy. Sessile polyp fragments that are left behind after incomplete resection will grow into new polyps and carry risks for malignancy.

In the recent past, endoscopic mucosal resection (EMR) has been adopted to remove sessile polyps. EMR involves the use of an injection to elevate surrounding mucosa followed by opening of a snare to cut the polyp and lastly use of biopsy forceps or a retrieval device to remove the polyp. The introduction and removal of the injection needle and snare through the length of the colonoscope, which is approximately 5.2 feet, must be repeated for the forceps.

The present disclosure relates to an endoscopic tool that is capable of delivering an innovative alternative to existing polyp removal tools, including snares, hot biopsy and EMR, by introducing a flexible powered instrument that that works with the current generation colonoscopes and can cut and remove any polyp. The endoscopic tool described herein can be designed to enable physicians to better address sessile or large polyps as well as remove multiple polyps in significantly less time. Through the adoption of the endoscopic tool described herein, physicians can become more efficient at early diagnosis of colorectal cancer.

The present disclosure will be more completely understood through the following description, which should be read in conjunction with the drawings. In this description, like numbers refer to similar elements within various embodiments of the present disclosure. Within this description, the claims will be explained with respect to embodiments. The skilled artisan will readily appreciate that the methods, apparatus and systems described herein are merely exemplary and that variations can be made without departing from the spirit and scope of the disclosure.

### A. Endoscopic Instrument

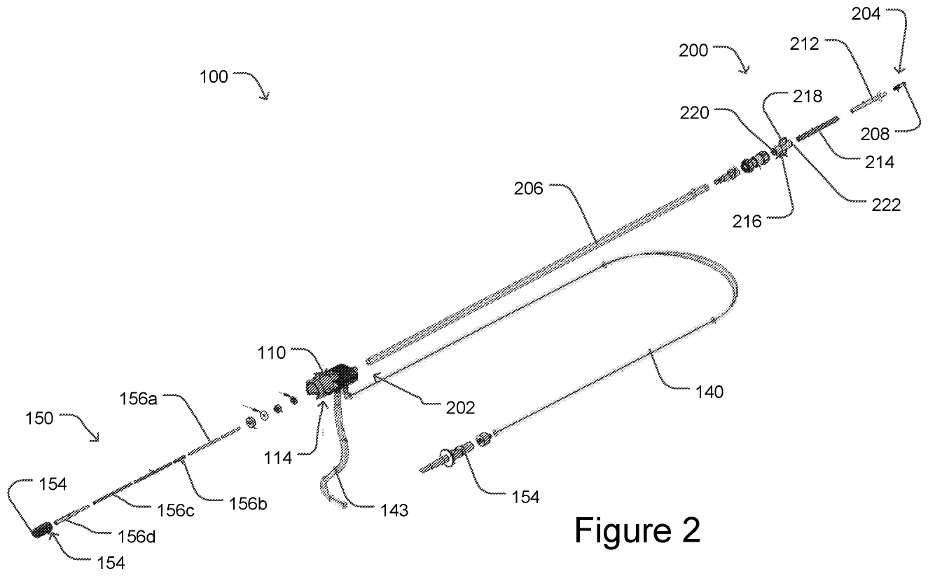

Referring back to the drawings, FIG. 2 illustrates an endoscopic tool 100 (e.g., endoscopic instrument) according to embodiments of the present disclosure. The endoscopic tool 100 may be similar to various endoscopic tools described in U.S. patent application Ser. No. 15/459,870, which is incorporated herein by reference in its entirety.

The endoscopic tool 100 can be configured to obtain samples of polyps and neoplasms from a patient. The endoscopic tool 100 can be configured to be rotated by a torque source (e.g., a motor coupled to a drive assembly or drive shaft of the endoscopic tool 100). The endoscopic tool 100 can be configured to flow irrigation fluid out into a site within a subject (e.g., a site within a colon, esophagus, lung of the subject). The endoscopic tool 100 can be configured to resect material at a site within a subject. The endoscopic tool 100 can be configured to provide a suction force via an aspiration channel to obtain a sample of the material resected at a site within a subject. In some implementations, the endoscopic tool 100 can be configured to be inserted within an instrument channel, such as an instrument channel of an endoscope (e.g., a gastroscope, such as a colonoscope, a laryngoscope, or any other flexible endoscope).

The endoscopic tool 100 includes a proximal connector 110 and a flexible torque delivery assembly 200. The proximal connector 110 is configured to couple a drive assembly 150 (e.g., a drive assembly including a drive shaft configured to be rotated by a source of rotational energy) of the endoscopic tool 100 to the flexible torque delivery assembly of the endoscopic tool 100. In some implementations, the proximal connector 110 includes a first connector end 114 at which the drive assembly 150 is coupled, and a second connector end 118 at which the flexible torque delivery assembly 200 is coupled. The first connector end 114 can include an inner wall 116 defining an opening in which the drive assembly 150 can be received. For example, in some implementations, the proximal connector 110 can be used to connect the drive assembly 150 to a drive shaft of a surgical console. The proximal connector 110 includes a drive transfer assembly 122. The drive transfer assembly 122 is configured to be operatively coupled to the drive assembly 150, receive torque from the drive assembly 150 when the drive assembly 150 rotates, and transfer the torque to the flexible torque delivery assembly 200 in order to rotate the flexible torque delivery assembly 200. In some implementations, the drive assembly 150, drive transfer assembly 122, and at least a portion of the flexible torque delivery assembly 200 are coaxial. For example, the drive transfer assembly 122 can be engaged to the drive assembly 150 along a drive axis 102, and the drive transfer assembly 122 can also be engaged to the flexible torque delivery assembly 200 at a proximal end 202 of the flexible torque delivery assembly 200 along the drive axis 102. It should be appreciated that rotating the flexible torque delivery assembly may include causing the flexible torque delivery assembly to rotate a component (such as an inner cannula) at one of the flexible torque delivery assembly.

In some implementations, the drive transfer assembly 122 includes gears, belts, or other drive components to control the direction and/or torque transferred from the drive assembly 150 to the flexible torque delivery assembly 200. For example, such drive components can be positioned at an angle to one another to change an axis of rotation of the flexible torque delivery assembly 200, or offset from one another to shift an axis of rotation of the flexible torque delivery assembly 200 relative to the drive axis 102.

In some implementations, the drive assembly 150 includes a drive engagement member 152. The drive engagement member 152 is configured to engage the drive assembly 150 to a source of rotational energy (e.g., a drive rotated by a motor, such as console drive assembly of a surgical console). The drive engagement member 152 can be configured to be fixedly and/or rigidly connected to the console drive assembly, such that the drive engagement member 152 rotates in unison with the console drive assembly. For example, as shown in FIG. 2, the drive engagement member 152 includes a proximal drive end 154 including a fitting (e.g., hex fitting, pin fitting, etc.) configured to engage (e.g., lock with, mate with, fixedly engage, frictionally engage, etc.) a console drive assembly. As such, rotation of the console drive assembly causes rotation of the drive engagement member 152.

In some implementations, the drive assembly 150 includes one or more shaft components 156 configured to transfer rotation of the drive engagement member 152 to the drive transfer assembly 122. In some implementations, the drive transfer assembly 122 includes the one or more shaft components 156. The shaft components 156 can include an insulator member 156a (e.g., a heat sheath, heat shrink, etc.) configured to insulate components of the drive assembly 150 from heat generated by rotation of the drive assembly or components thereof. The shaft components 156 can include a cutter 156b. The shaft components 156 can include a shaft torque coil 156c which may be similar to other torque coils described herein. In some implementations, the shaft components 156 can include a shaft torque rope. The shaft components 156 can include a shaft tube 156d. The shaft tube 156d can include a radius that is less than a relatively greater radius of the drive engagement member 152 (e.g., a relatively greater radius that may facilitate receiving rotational energy from a drive shaft or other rotational energy source, such as by engaging the drive engagement member 152 to a console drive assembly). For example, the shaft tube 156d can include a relatively lesser smaller corresponding more closely to a radius of the drive transfer assembly 122 and/or the flexible torque delivery assembly 200. In such implementations, the torque received at the drive transfer assembly 122 and/or the flexible torque delivery assembly 200 can be modified (e.g., increased) in a manner corresponding to the change in radius between the radius of the drive engagement member 152 and the radius of the shaft tube 156d.

In some implementations, the cutting assembly 201 can include an outer cannula and an inner cannula disposed within the outer cannula. The outer cannula can define an opening 208 through which material to be resected can enter the cutting assembly 201. In some implementations, the opening 208 is defined through a portion of the radial wall of the outer cannula. In some implementations, the opening 208 may extend around only a portion of the radius of the outer cannula, for example, up to one third of the circumference of the radial wall. As the aspiration channel extends between a vacuum port (e.g., vacuum port 126) and the opening 208, any suction applied at the vacuum port causes a suction force to be exerted at the opening 208. The suction force causes material to be introduced into the opening or cutting window of the outer cannula, which can then be cut by the inner cannula of the cutting assembly 201.

The inner cannula can include a cutting section that is configured to be positioned adjacent to the opening 208 such that material to be resected that enters the cutting assembly 201 via the opening 208 can be resected by the cutting section of the inner cannula. The inner cannula may be hollow and an inner wall of the inner cannula may define a portion of an aspiration channel that may extend through the length of the endoscopic tool. A distal end of the inner cannula can include the cutting section while a proximal end of the inner cannula can be open such that material entering the distal end of the inner cannula via the cutting section can pass through the proximal end of the inner cannula. In some implementations, the distal end of the inner cannula can come into contact with an inner surface of a distal end of the outer cannula. In some implementations, this can allow the inner cannula to rotate relative to the outer cannula along a generally longitudinal axis, providing more stability to the inner cannula while the inner cannula is rotating. In some implementations, the size of the opening can dictate the size of the materials being cut or resected by the inner cannula. As such, the size of the opening may be determined based in part on the size of the aspiration channel defined by the inner circumference of the flexible torque coil.

The endoscopic tool 100 can include a flexible torque coil 212 that is configured to couple to the proximal end of the inner cannula at a distal end of the flexible torque coil 212. The flexible torque coil can include a fine coil with multiple threads and multiple layers, which can transmit the rotation of one end of the flexible torque coil to an opposite end of the flexible torque coil. Each of the layers of thread of the flexible torque coil can be wound in a direction opposite to a direction in which each of the layers of thread adjacent to the layer of thread is wound. In some implementations, the flexible torque coil can include a first layer of thread wound in a clockwise direction, a second layer of thread wound in a counter-clockwise direction and a third layer of thread wound in a clockwise direction. In some implementations, the first layer of thread is separated from the third layer of thread by the second layer of thread. In some implementations, each of the layers of thread can include one or more threads. In some implementations, the layers of thread can be made from different materials or have different characteristics, such as thickness, length, among others.

The flexibility of the torque coil 212 allows the coil to maintain performance even in sections of the torque coil 212 that are bent. Examples of the flexible torque coil 212 include torque coils made by ASAHI INTECC USA, INC located in Santa Ana, Calif., USA. In some implementations, the flexible torque coil 212 can be surrounded by a sheath or lining (e.g., sheath 214) to avoid frictional contact between the outer surface of the flexible torque coil 212 and other surfaces. In some implementations, the flexible torque coil 212 can be coated with Polytetrafluoroethylene (PFTE) to reduce frictional contact between the outer surface of the flexible torque coil 212 and other surfaces. The flexible torque coil 212 can be sized, shaped or configured to have an outer diameter that is smaller than the diameter of the instrument channel of the endoscope in which the endoscopic tool is to be inserted. For example, in some implementations, the outer diameter of the flexible torque coil can be within the range of 1-4 millimeters. The length of the flexible torque coil can be sized to exceed the length of the endoscope. In some implementations, the inner wall of the flexible torque coil 212 can be configured to define another portion of the aspiration channel that is fluidly coupled to the portion of the aspiration channel defined by the inner wall of the inner cannula of the cutting assembly 201. A proximal end of the flexible torque coil 212 can be coupled to the proximal connector 110 (e.g., to the drive transfer assembly 122 of the proximal connector 110, etc.).

The endoscopic tool 100 can include a flexible outer tubing 206 that can be coupled to the proximal end of the outer cannula. In some implementations, a distal end of the flexible outer tubing 206 can be coupled to the proximal end of the outer cannula using a coupling component. In some implementations, the outer cannula can be configured to rotate responsive to rotating the flexible outer tubing. In some implementations, the flexible outer tubing 206 can be a hollow, braided tubing that has an outer diameter that is smaller than the instrument channel of the endoscope in which the endoscopic tool 100 is to be inserted. In some implementations, the length of the flexible outer tubing 206 can be sized to exceed the length of the endoscope. The flexible outer tubing 206 can define a bore through which a portion of the flexible outer tubing 206 extends. The flexible outer tubing 206 can include braids, threads, or other features that facilitate the rotation of the flexible outer tubing 206 relative to the flexible torque coil, which is partially disposed within the flexible outer tubing 206. The flexible outer tubing can define a portion of an irrigation channel for outputting fluid to a site within a subject.

The endoscopic tool 100 can include a rotational coupler 216 configured to be coupled to a proximal end of the flexible outer tubing 206. The rotational coupler 216 may be configured to allow an operator of the endoscopic tool to rotate the flexible outer tubing 206 via a rotational tab 218 coupled to or being an integral part of the rotational coupler 216. By rotating the rotational tab 218, the operator can rotate the flexible outer tubing and the outer cannula along a longitudinal axis of the endoscope and relative to the endoscope and the inner cannula of the cutting assembly 201. In some implementations, the operator may want to rotate the outer cannula while the endoscopic instrument is inserted within the endoscope while the endoscope is within the patient. The operator may desire to rotate the outer cannula to position the opening of the outer cannula to a position where the portion of the radial wall of the outer cannula within which the opening is defined may aligned with the camera of the endoscope such that the operator can view the material entering the endoscopic instrument for resection via the opening. This is possible in part because the opening is defined along a radial wall extending on a side of the outer cannula as opposed to an opening formed on the axial wall of the outer cannula.

In some implementations, a proximal end 220 of the rotational coupler 216 can be fluidly coupled to the proximal connector 110, such that the irrigation channel of the endoscopic tool 100 passes from an irrigation port 134 through the flexible outer tubing 206 into the rotational coupler 216. Irrigation fluid entering the proximal connector 110 at the irrigation port 134 can thus pass through the rotational coupler 216 in order to be outputted at a site within a subject. In some implementations, the rotational coupler 216 can be a rotating luer component that allows a distal end 222 of the rotational coupler 216 to rotate relative to the proximal end 220 of the rotational coupler 216. In this way, when the flexible outer tubing 206 is rotated, the component to which the proximal end of the rotational coupler 216 is coupled, is not caused to rotate. The rotational coupler 216 can define a bore along a central portion of the rotational coupler 216 through which a portion of the flexible torque coil 212 extends. In some implementations, the rotational coupler 216 can be a male to male rotating luer connector. In some implementations, the rotational coupler can be configured to handle pressures up to 1200 psi.

In some implementations, the flexible torque delivery assembly 200 is configured to be fluidly coupled to a vacuum source to apply a suction force to the aspiration channel. The aspiration channel allows for fluid and material (e.g., a sample to be obtained) to be drawn into the distal end 204 of the flexible torque delivery assembly 200 in order to flow to the proximal end 202 of the flexible torque delivery assembly 200. For example, after the cutting assembly 201 has been used to resect material from a site within a subject, vacuum pressure can be applied through the aspiration channel to draw (e.g., transfer by suction, etc.) fluid and material into the flexible torque delivery assembly 200.

In some implementations, the proximal connector 110 is configured to be coupled to a vacuum source to provide a suction force for aspiration. For example, as shown in FIG. 2, the proximal connector 110 includes a vacuum port 126 (e.g., aspiration port). The vacuum port/aspiration port 126 can be similar to other aspiration ports disclosed herein. The vacuum port 126 is configured to fluidly couple an aspiration channel of the endoscopic tool 100 to a vacuum source (e.g., to a vacuum source with a specimen receiver positioned between the vacuum source and the endoscopic tool). The vacuum port 126 is configured to transmit a suction force applied to the vacuum port 126 to the aspiration channel, in order to draw fluid and material entering the distal end 204 of the endoscopic tool 100 through the aspiration channel towards the vacuum source. In some implementations, such as shown in FIG. 2, the vacuum port 126 includes a vacuum port channel 130 oriented transverse to the drive axis 102 (and thus the aspiration channel). This may facilitate coupling tubing to the vacuum port 126 that extends to a specimen receiver or vacuum source without interfering with manipulation of the proximal connector 110 and the endoscopic tool 100. In various implementations, the vacuum port channel 130 can be oriented at varying angles relative to the drive axis 102. In some implementations, vacuum tubing 132 can be coupled to the vacuum port 126.

In some implementations, the proximal connector 110 is configured to be coupled to a fluid source to provide fluid to be outputted by the endoscopic tool 100 to a site within a subject. As shown in FIG. 2, the proximal connector 110 includes an irrigation port 134, including an irrigation port channel 136, configured to receive fluid from a fluid source. The irrigation port 134 is configured to be fluidly coupled to an irrigation channel of the flexible torque delivery assembly 200 (e.g., an irrigation channel defined between the flexible outer tubing 206 and the flexible torque coil 212 and extending to an opening at the distal end 204 of the flexible torque delivery assembly 200), such that fluid can flow from the proximal connector 110 through flexible torque delivery assembly 200 to be outputted at a site within a subject. In some implementations, the fluid (e.g., irrigation fluid) can be used to cool the flexible torque delivery assembly 200, which may generate heat due to friction caused by rotation or other movements. In some implementations, the fluid can be used to wash a site within a subject. In some implementations, the fluid provides lubrication to facilitate rotation or other movement of components of the endoscopic tool 100 relative to one another. In some implementations, the irrigation port 134 is configured to be coupled to a fluid transfer device or irrigation pump. The irrigation port 134 receives a flow of irrigation fluid from the irrigation pump and transfers the fluid into the irrigation channel. In some implementations, the irrigation channel is defined to include the irrigation port 134 and/or tubing connecting the irrigation port 134 to the fluid source. In some implementations, the irrigation port 134 can be coupled to a fluid source by fluid tubing 140. The fluid tubing 140 can be coupled to a fitting 144 (e.g., vented spike fitting, non-vented spike fitting, etc.) configured to interface the fluid tubing 140 to a fluid source.

B. Systems and Methods of Endoscopic Instruments with Articulating End

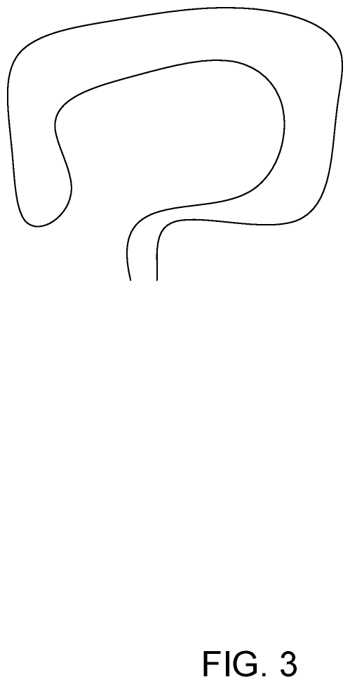

In existing endoscopic tool systems, a cutting assembly may be provided which can be rotated to resect polyps and other materials from a site within a subject. However, in certain use cases or procedures, the cutting assembly may not be able to effectively resect desired material, such as to resect relatively large portions of polyps adjacent to where the polyps protrude from underlying tissue. For example, tortuous pathways in the colon (see FIG. 3), pancreas, or duodenum may require a catheter to make several high-angle turns to reach a sample site; attempting to further change an angle of an endoscopic instrument extending out of the catheter can limit the ability to rotate portions of a cutting assembly of the endoscopic instrument, such as by damaging or breaking a flexible torque coil.

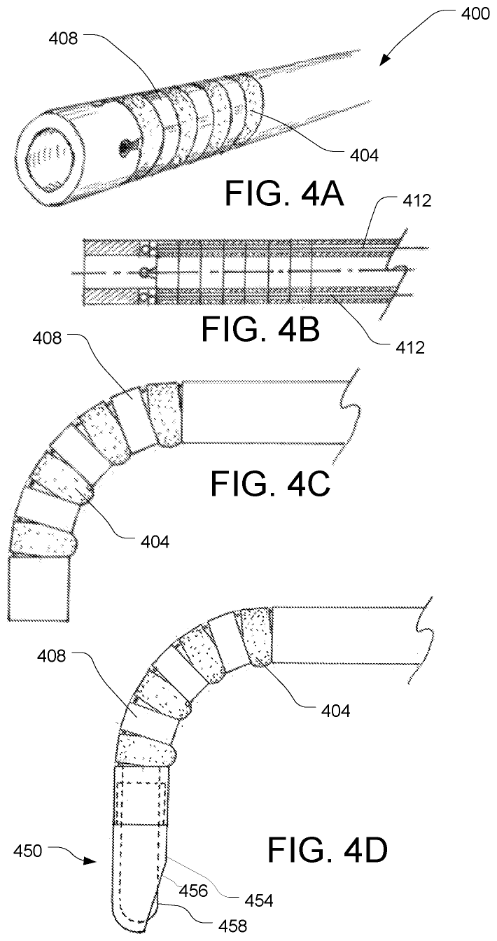

Referring generally to FIGS. 4A-9, an endoscope system can include an endoscope and an endoscopic instrument. The endoscope can include an instrument channel, an external sheath coupled to the endoscope, or both. The endoscopic instrument can be inserted through at the instrument channel or the external sheath. The endoscopic instrument can include an outer tubing extending from a proximal tubing end to a distal tubing end. The endoscopic instrument can include a flexible torque component extending through the outer tubing. The flexible torque component can include at least one of a flexible torque wire or a flexible torque coil. The articulation assembly can be coupled to the distal tubing end of the outer tubing, and can include a plurality of segments and at least one control member extending from a proximal control end to a distal control end coupled with the plurality of segments. The at least one control member can manipulate an orientation of at least one segment of the plurality of segments responsive to receiving a control input at the proximal control end. The endoscopic instrument can include an end effector coupled to a distal end of the flexible torque component such that manipulating the orientation of the at least one segment controls an orientation of the end effector relative to the longitudinal axis of the endoscope and rotation of the flexible torque component controls an angle of rotation of the end effector relative to an effector axis of the end effector. Manipulating the orientation may include manipulating a pose (e.g., position and orientation) of the end effector. As such, the endoscopic instrument can be used to articulate the end effector to a greater range of orientations and positions, even after passing through a tortuous pathway.

Referring now to FIGS. 4A-4C, an articulation assembly 400 (e.g., articulation assembly) of an endoscopic instrument (e.g., endoscopic instrument 312) is illustrated according to an embodiment of the present disclosure. The articulation assembly 400 can be proximal to a cutting assembly of the endoscopic instrument and/or be disposed around at least a portion of the cutting assembly. The articulation assembly 400 can be coupled to a distal end of outer tubing (e.g., outer tubing 206), so that rotation of the outer tubing causes rotation of the articulation assembly 400.

The articulation assembly 400 includes a plurality of first segments 404 alternating with a plurality of second segments 408. The first segments 404 can be compliant, while the second segments 408 are non-compliant. For example, the second segments 408 can have a rigidity greater than a threshold rigidity. The articulation assembly 400 includes a plurality of control wires 412 extending through each of the segments 404, 408. In some embodiments, the articulation assembly 400 includes four control wires 412, each spaced approximately equidistantly around a circumference of the articulation assembly 400. The control wires 412 may directly or indirectly be controlled from a proximal end of the endoscopic instrument that includes the articulation assembly 400. In some embodiments, the threshold rigidity corresponds to a nominal tension or maximum tension that can be applied to the control wires 412, such that the first segments 404 comply with (e.g., can be compressed or otherwise modified in shape) tension applied by the control wires 412, while the second segments 408 do not comply with the applied tension. As shown in FIG. 4C, applying tension to one or more of the control wires 412 can selectively articulate the articulation assembly 400 in a direction corresponding to how the one or more control wires 412 are controlled. As such, two-axis articulation can be enabled. As shown in FIG. 4D, articulation of the articulation assembly 400 can articulate a cutting assembly 450 (e.g., a cutting assembly similar to cutting assembly 201) including an outer cutter 454 and an inner cutter 458, which can enable the cutting assembly 450 to more effectively reach remote sample sites while reducing risk of damage to or reduced functionality of components of the endoscopic instrument. For example, the outer cutter 454 can be coupled to a distal-most segment 404 (or distal-most segment 408, depending on the number and ordering of the segments 404, 408), such that articulation of the articulation assembly 400 enables articulation of the outer cutter 454, including manipulation of a cutting window 456 defined by the outer cutter 454. Depending on how the articulation assembly 400 is controlled, the cutting assembly 450 may be manipulated while the articulation assembly 400 is articulated (e.g., changed in orientation in one or more axes) or at different times.

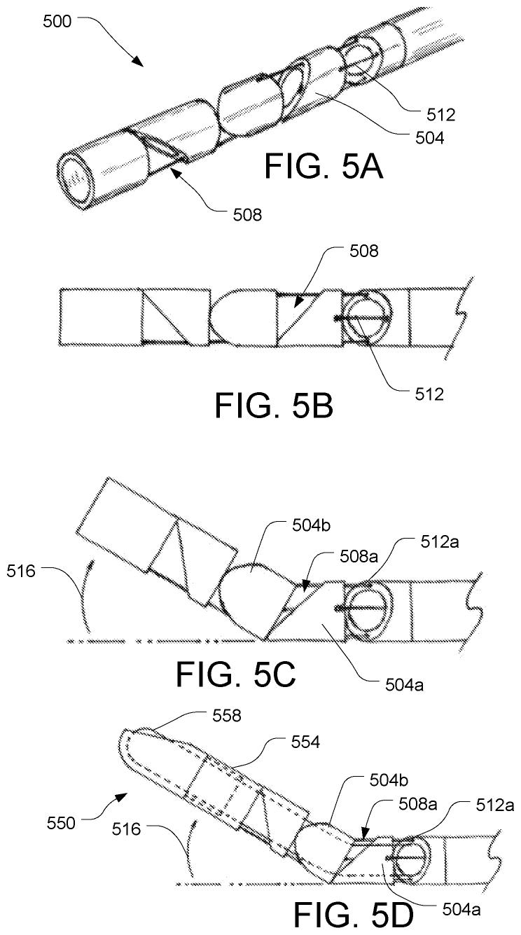

Referring now to FIGS. 5A-5C, an articulation assembly 500 of an endoscopic instrument is shown. The articulation assembly 500 can incorporate features of the articulation assembly 400. The articulation assembly 500 includes a plurality of alternating angled segments 504, such that a gap 508 is provided between each pair of adjacent segments 504. A direction of each gap 508 can correspond to a relative position of each of a plurality of control wires 512 extending through the plurality of segments 504. As such, when a particular control wire 512 is put under tension, a corresponding pair of segments 504 will move towards one other due to the gap 508 between those segments 504. For example, applying tension to the control wire 512a causes the segments 504a, 504b to move towards one another in the space of the gap 508a, causing the articulation assembly 500 to articulate in a corresponding direction 516. As shown in FIGS. 5A-5C, the plurality of segments 504 may include four segments 504, each corresponding to a respective control wire 512 of four control wires 512. As such, the articulation assembly 500 can enable two-axis articulation. As shown in FIG. 5D, articulation of the articulation assembly 500 can articulate a cutting assembly 550 including an outer cutter 554 and an inner cutter 558, which can enable the cutting assembly 550 to more effectively reach remote sample sites while reducing risk of damage to or reduced functionality of components of the endoscopic instrument.

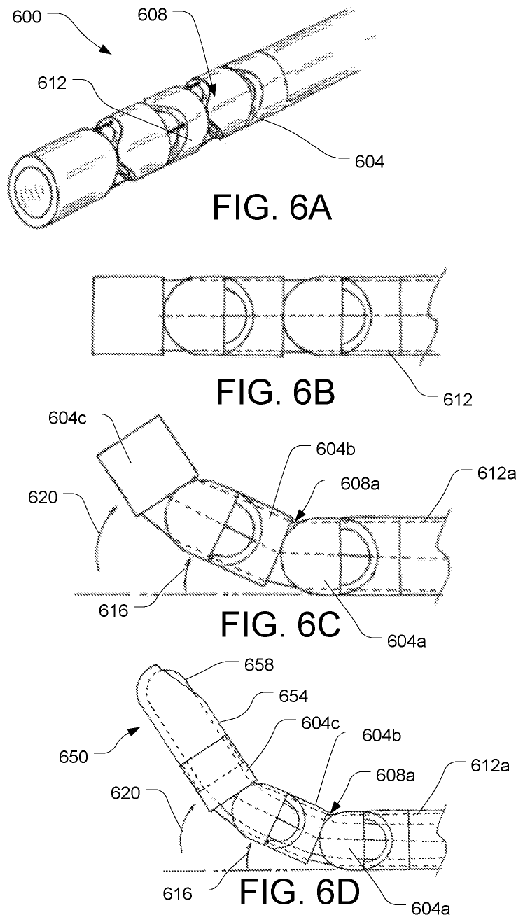

Referring now to FIGS. 6A-6C, an articulation assembly 600 of an endoscopic instrument is shown. The articulation assembly 600 can incorporate features of the articulation assemblies 400, 500. The articulation assembly 600 includes a plurality of alternating radiused segments 604, such that a gap 608 is provided between each pair of adjacent segments 604. Similar to the articulation assembly 500, selectively controlling each of a plurality of control wires 612 can enable two-axis directional control of articulation 616 of the articulation assembly 600. As shown in FIGS. 6A-6C, each segment 604 can be shaped to provide multiple gaps 608 between adjacent segments 604, such that, as shown in FIG. 6C, applying tension to control wire 612a causes segment 604a and 604b to move relative to one another and also causes segments 604b and 604c to move relative to one another to enable articulation 616 and articulation 620; selection of control wire 612 to which tension is applied can enable two-axis articulation. As shown in FIG. 6D, articulation of the articulation assembly 600 can articulate a cutting assembly 650 including an outer cutter 654 and an inner cutter 658, which can enable the cutting assembly 650 to more effectively reach remote sample sites while reducing risk of damage to or reduced functionality of components of the endoscopic instrument.

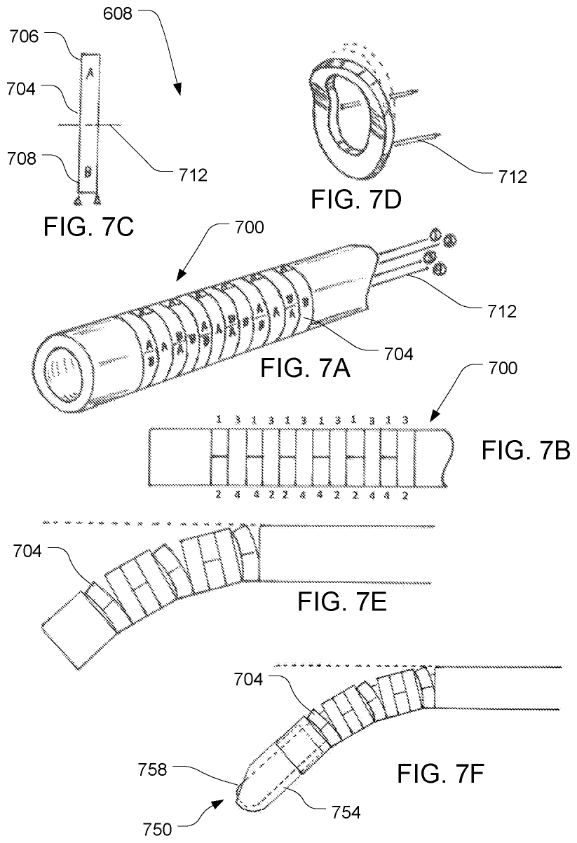

Referring now to FIGS. 7A-7E, an articulation assembly 700 is shown. The articulation assembly 700 can incorporate features of the articulation assemblies 400, 500, 600. The articulation assembly 700 includes a plurality of segments 704 each having a first end 706 and a second end 708. The segments 704 can be made of an electrically articulated material, such as an electroactive polymer and/or electromagnetic coils disposed about a flexible material. The segments 704 can be arranged to alternate in orientation such that adjacent segments 704 are oriented approximately 90 degrees relative to one another, as shown in FIG. 7A. The articulation assembly 700 includes a plurality of conductors 712 (e.g., conducting wires) extending through the plurality of segments 704. For example, the articulation assembly 700 is shown to include four conductors 712 (corresponding to the four possible orientations of the segments 704). As shown in FIG. 7B, the conductors 712 can be coupled in a predetermined order to specific segments 704; for example, conductor 1 can be coupled to alternating segments 704 starting from a distal segment 704, conductor 3 can be coupled to alternating segments 704 starting from a proximal segment 704, and conductors 2 and 4 can be coupled to pairs of adjacent segments 704. As such, each possible pair of potential conductors 712 (e.g., 1 and 2; 1 and 4; 2 and 3; 3 and 4) can be coupled to predetermined segments 704 (e.g., in an alternating sequence over four adjacent segments 704), such that by selectively actuating a particular pair of conductors 712, particular segments 704 can be caused to change in shape as shown in FIG. 7E, enabling the articulation assembly 700 to articulate in two axes. As shown in FIG. 7F, articulation of the articulation assembly 700 can articulate a cutting assembly 750 including an outer cutter 754 and an inner cutter 758, which can enable the cutting assembly 750 to more effectively reach remote sample sites while reducing risk of damage to or reduced functionality of components of the endoscopic instrument.

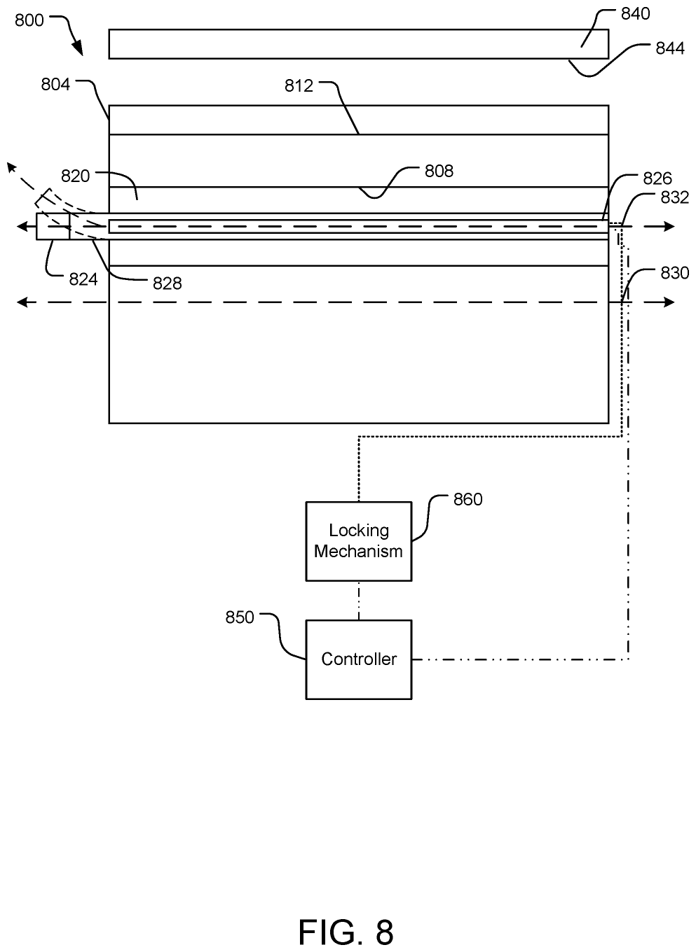

Referring now to FIG. 8, an endoscope system 800 is shown. The endoscope system 800 can incorporate features of the endoscopic tool 100, the articulation assembly 400, the articulation assembly 500, the articulation assembly 600, he articulation assembly 700, or various combinations thereof.

The endoscope system 800 includes an endoscope 804. The endoscope 804 can be provided to a site within a subject (e.g., through the colon of the subject depicted in FIG. 3) to allow an endoscopic instrument 820 to be positioned at the site within the subject, such as to remove material from the site within the subject.

The endoscope 804 may include an instrument channel 808 (e.g., working channel) that is defined within the endoscope 804 and extends from a proximal channel end (which may remain outside of the subject) to a distal channel end (which may be provided to the site within the subject). The endoscopic instrument 820 may be provided through the instrument channel 808 so that a distal end of the endoscopic instrument 820 can extend out of the instrument channel 808.

The endoscope 804 may include various other channels and components. For example, the endoscope 804 may include an image capture device (e.g., camera), light sources, irrigation channels, or any combination thereof.

A sheath 840 may be coupled to the endoscope 804. For example, the sheath 840 may be coupled to the endoscope 804 to at extend at least partially around an outer surface 812 of the endoscope 804, such that the sheath 840 is external to the endoscope 804. The sheath 840 may define a sheath channel 844, which may be of similar size to the instrument channel 808. The endoscope system 800 may include one or both of the instrument channel 808 or the sheath 840.

As described above with respect to various articulation assemblies, the endoscopic instrument 820 can be configured so that an end effector 824 (e.g., snare, scissors, clamp, cutting assembly) at the distal end of the endoscopic instrument 820 can extend out of the instrument channel 808, and responsive to operation of an articulation assembly 828, an orientation of the end effector 824 can be manipulated in multiple axes. For example, the orientation can be manipulated in a first axis 832 perpendicular to longitudinal axis 830 (e.g., to move the end effector 824 up or down relative to a frame of reference defined with respect to longitudinal axis 830, such as depicted with respect to various articulation assemblies in FIGS. 4C, 4D, 5C, 5D, 6C, 6D, 7E, and 7F) or a second axis (not shown, extending into the page, to move the end effector 824 left or right relative to the frame of reference defined with respect to longitudinal axis 830). The orientation can be manipulated to change an orientation of a cutting window or suction window (e.g., cutting window 456, a suction window defined at a distal end of inner cutter 458) of the end effector 824. As shown in FIG. 8, by providing the end effector 824 through the sheath channel 844, the end effector 824 may be able to access a greater range of positions for reaching material at the site within the subject.

The endoscopic instrument 824 can define a suction channel 826 that extends from the proximal end of the endoscopic instrument 824 to the distal end of the endoscopic instrument. For example, the suction channel 826 can have an inlet at the end effector 824, such that a vacuum force applied to an outlet of the suction channel 826 at the proximal end of the endoscopic instrument can be used to remove material from the site within the subject through the suction channel 826. The suction channel 826 can extend through outer tubing of the endoscopic instrument 824.

In some embodiments, the articulation assembly 828 (e.g., control members of the articulation assembly 828) can be manually manipulated. For example, proximal ends of the articulation assembly 828 can be put under tension (e.g., pulled) to manipulate the articulation assembly 828.

In some embodiments, the endoscope system 800 includes a controller 850. The controller 850 can include one or more processors and a memory. The one or more processors may be implemented as a specific purpose processor, an application specific integrated circuit (ASIC), one or more field programmable gate arrays (FPGAs), a system on a chip (SoC), a group of processing components (e.g., multicore processor), or other suitable electronic processing components. The memory is one or more devices (e.g., RAM, ROM, flash memory, hard disk storage) for storing data and computer code for completing and facilitating the various user or client processes, layers, and modules described in the present disclosure. The memory may be or include volatile memory or non-volatile memory and may include database components, object code components, script components, or any other type of information structure for supporting the various activities and information structures of the inventive concepts disclosed herein. The memory is communicably connected to the processor and includes computer code or instruction modules for executing one or more processes described herein. The memory includes various circuits, software engines, and/or modules that cause the processor to execute the systems and methods described herein. The controller 850 may be implemented using a surgical console.

The controller 850 can receive control inputs for controlling the articulation assembly 828 as well as the end effector 824 (e.g., via a user interface of the surgical console). For example, the controller 850 can receive a first control input indicating a target value for the orientation of the end effector 824, and a second control input indicative of instructions to drive the end effector 824 (e.g., rotate the end effector 824, move the end effector 824 along longitudinal axis 830). The controller 850 can drive one or more actuators (e.g., linear actuators, motors) coupled to the control members of the articulation assembly 828 to control the orientation of the end effector 824. The controller 850 can drive one or more actuators (e.g., control drive assembly 150) to rotate drive a flexible torque component coupled to the end effector 824 to drive the end effector 824. This can enable the articulation assembly 828 and end effector 824 to be simultaneously and independently manipulated, such as to sweep the end effector 824 over a range of positions while manipulating (e.g., rotating) the end effector 824, or to move the end effector 824 into position prior to rotating the end effector 824.

In some embodiments, the endoscope system 800 includes a locking mechanism 860 coupled to the articulation assembly 828. The locking mechanism 860 can restrict movement of the articulation assembly 828 (e.g., control members of the articulation assembly 828) to set the end effector 824 to a target orientation. For example, the locking mechanism 860 can one or more clamps, gears, brakes, or any combination thereof that can selectively contact one or more of the control members of the articulation assembly 828 to prevent the selected one or more control members from moving. In some embodiments, the locking mechanism 860 can be operated to selectively restrict movement of the articulation assembly 828 to a single degree of freedom (e.g., lock movement of the articulation assembly 828 in the first axis 832 while allowing movement in the second axis). The locking mechanism 860 can be coupled to a motor that spools or unspools control wires of the articulation assembly 828 to lock the articulation assembly 828. In some embodiments, the controller 850 controls operation of the locking mechanism 860, such as in response to an input indicating instructions to lock or unlock the articulation assembly 828. In some embodiments, the controller 850 performs an interlock function (which can be selectively activated or deactivated) with respect to the locking mechanism 860 and the end effector 824. For example, the controller 850 can set the locking mechanism 860 to a lock state to lock the articulation assembly 828 responsive to receiving instructions to drive the end effector 824, which can facilitate automatic stabilization of the end effector 824 at the target orientation when removing material from the site within the subject.

Accordingly, through operation of various components of the endoscope system 800, each of the following manipulations of the end effector 824 can be performed independently and concurrently or at different times: rotation of a flexible torque component coupled to the end effector 824 to drive the end effector 824, such as to rotate an inner cutter of the end effector 824 to remove material at the site within the subject; articulation of the articulation assembly 828 in one or more selected axes, enabling three hundred sixty degree manipulation of the position of the end effector 824; locking of the articulation assembly 828 in one or more degrees of freedom; rotation of the cutting window of the end effector 824 (e.g., of an outer cutter of the end effector 824, such as through rotation of outer tubing 206 using rotational coupler 216).

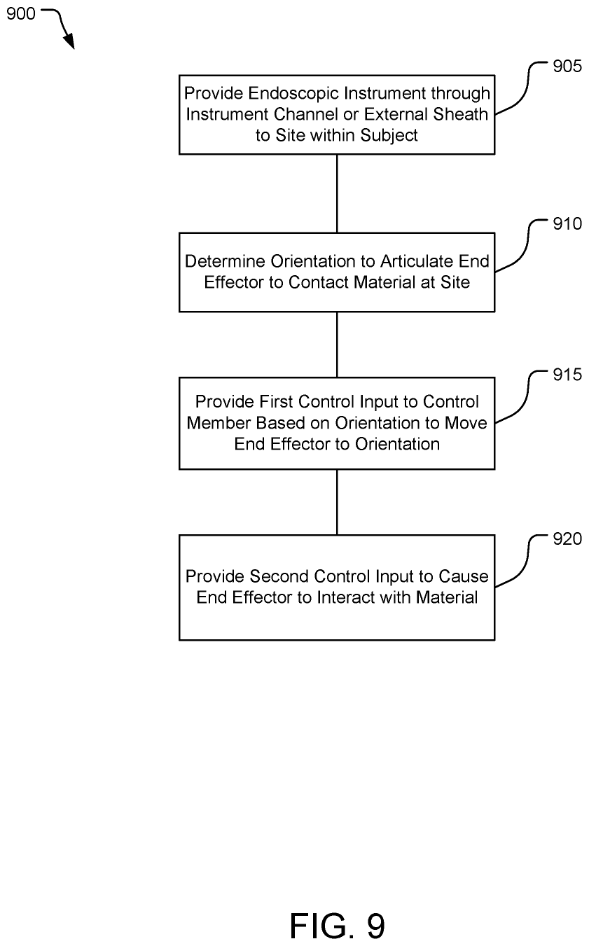

Referring now to FIG. 9, a method 900 for controlling operation of an endoscopic instrument is shown. The method 900 can be performed using various devices and system described herein, including the endoscopic tool 100, the articulation assemblies 400, 500, 600, 700, and the endoscope system 800. The method 900 can be performed by an operator (e.g., medical professional) controlling the endoscopic instrument, including by operating a surgical console or other control device coupled with components of the endoscopic instrument. The method 900 can be performed by controlling components coupled to the endoscopic instrument disposed outside of a subject while the endoscopic instrument is provided to a site within the subject, such as a site at which material to be removed from the subject is located.

At 905, the endoscopic instrument is provided to the site within the subject. The site may be identified using an image capture device of an endoscope or based on other imaging modalities (e.g., ultrasound, CT, X-ray, MRI). The endoscopic instrument can be provided to the site by being moved through an instrument channel of the endoscope, or by being moved through an external sheath coupled to the endoscope. The endoscopic instrument can be moved to a position at which an articulation assembly coupled with an end effector of the endoscopic instrument extends out from the endoscope (e.g., out from the instrument channel or external sheath) towards the site within the subject.

At 910, at least one of a position or orientation (e.g., pose) to articulate the end effector to contact the end effector to material at the site within the subject is determined. The at least one of the position or orientation can be determined based on images detected by the image capture device or other imaging modalities. The at least one of the position or the orientation may outside of a nominal range defined by an outer surface of the endoscope (e.g., beyond a cylindrical shell extending from the edges of the endoscope) such that as described further herein, the endoscopic instrument can be moved through a greater range of positions and orientations.

At 915, a first control input is provided to at least one control member of an articulation assembly to cause the control member to move the end effector to the determined at least one of position or orientation. For example, the first control input can include manual manipulation of control wires of the articulation assembly coupled to a plurality of segments of the articulation assembly. The first control input can include instructions to manipulate the control wires, such as to use a motor or other actuator to increase or decrease tension applied to one or more selected control wires, or to provide an electrical current through the one or more selected control wires. In some embodiments, the first control input can be provided in an iterative manner to adjust the at least one of the position or the orientation until a target position or orientation is achieved. In some embodiments, a cutting window of the end effector is manipulated, such as by rotating a rotational coupler coupled to an outer tubing coupled to the articulation assembly, which may be coupled to an outer cutter of the end effector defining the cutting window. The first control input can be used to control the articulation assembly in any of one or more selected axes of rotation, enabling three hundred sixty degree range of motion of the end effector.

At 920, a second control input is provided to cause the end effector to interact with the material. For example, the second control input can be provided to cause a drive assembly to rotate a flexible torque component extending through the endoscopic instrument and coupled with the end effector in order to manipulate the end effector (e.g., rotate the end effector about a longitudinal axis of the end effector; cause a linear actuator to move the end effector along the longitudinal axis). The second control input can be used to cause the end effector to move simultaneously with operating the articulation assembly or separately from operating the articulation assembly.

In some embodiments, a locking mechanism can be activated to lock the articulation assembly to a target pose, such as to lock the articulation assembly in place while using the end effector to remove material from the site within the subject. In some embodiments, the locking mechanism is activated responsive to the second control input, enabling an interlock function between driving of the end effector and movement of the articulation assembly. In some embodiments, the locking mechanism is activated responsive to input that drives a vacuum source to apply a vacuum to a suction channel of the endoscopic instrument.

The construction and arrangement of the systems and methods as shown in the various exemplary embodiments are illustrative only. Although only example embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. An endoscope system, comprising:
an endoscope comprising at least one of an instrument channel or an external sheath coupled to the endoscope, the endoscope defining a longitudinal axis along which the at least one of the instrument channel or the external sheath extends; and
an endoscopic instrument configured to be inserted through the at least one of the instrument channel or the external sheath, the endoscopic instrument comprising:
an outer tubing extending from a proximal tubing end to a distal tubing end;
a flexible torque component extending through the outer tubing, the flexible torque component comprising at least one of a flexible torque wire or a flexible torque coil;
an articulation assembly coupled to the distal tubing end of the outer tubing, the articulation assembly comprising a plurality of segments and at least one control member extending from a proximal control end to a distal control end coupled with the plurality of segments, the at least one control member configured to manipulate an orientation of at least one segment of the plurality of segments responsive to receiving a control input at the proximal control end; and
an end effector coupled to a distal end of the flexible torque component such that manipulating the orientation of the at least one segment controls an orientation of the end effector relative to the longitudinal axis of the endoscope and rotation of the flexible torque component controls an angle of rotation of the end effector relative to an effector axis of the end effector.

2. The endoscope system of claim 1, wherein the at least one control member is configured to manipulate the orientation of the at least one segment by manipulating at least two degrees of freedom of the plurality of segments relative to the longitudinal axis of the endoscope.

3. The endoscope system of claim 1, wherein the end effector comprises at least one of a snare or a cutting member that extends beyond an outlet of the at least one of the instrument channel of the external sheath.

4. The endoscope system of claim 1, further comprising an outer cutter coupled to a distal end of the articulation assembly, the outer cutter defining a cutting window, wherein the end effector comprises an inner cutter configured to rotate within the outer cutter responsive to actuation of the flexible torque component by a drive assembly coupled to a proximal end of the flexible torque component.

5. The endoscope system of claim 4, wherein the at least one control member is configured to control an orientation of the cutting window relative to the longitudinal axis of the endoscope.

6. The endoscope system of claim 1, further comprising a suction channel extending through the outer tubing from a proximal suction end to a distal suction end proximate the end effector such that material received by the end effector moves through the suction channel from the distal suction end to the proximal suction end responsive to a vacuum force applied to the proximal suction end.

7. The endoscope system of claim 1, wherein the external sheath extends around an outer wall of the endoscope.

8. The endoscope system of claim 1, further comprising one or more processors configured to receive a first control input indicative a target value for the orientation of the end effector, receive a second control input indicative of instructions to rotate the end effector, and cause the at least one control member to manipulate the at least one segment while causing the flexible torque component to rotate the end effector responsive to the first control input and the second control input.

9. The endoscope system of claim 1, further comprising a locking assembly configured to restrict movement of the at least one control member to set the articulation assembly to a predetermined orientation.

10. The endoscope system of claim 1, wherein the at least one control member comprises a plurality of control wires.

11. The endoscope system of claim 1, wherein the plurality of segments each comprise at least one of an electroactive polymer, a shape memory alloy, an electromagnetic coil, or a permanent magnet, and the at least one control member is configured to output at least one of an electrical field or a magnetic field to control a shape of the plurality of segments.

12. An endoscopic instrument configured to be inserted through at least one of an instrument channel of an endoscope or an external sheath of the endoscope, the endoscopic instrument comprising:
an outer tubing extending from a proximal tubing end to a distal tubing end;
a flexible torque component extending through the outer tubing, the flexible torque component comprising at least one of a flexible torque wire or a flexible torque coil;
an articulation assembly coupled to the distal tubing end of the outer tubing, the articulation assembly comprising a plurality of segments and at least one control member extending from a proximal control end to a distal control end coupled with the plurality of segments, the at least one control member configured to manipulate an orientation of at least one segment of the plurality of segments responsive to a control input at the proximal control end; and
an end effector coupled to a distal end of the flexible torque component such that manipulating the orientation of the at least one segment controls an orientation of the end effector relative to the outer tubing and rotation of the flexible torque component controls an angle of rotation of the end effector relative to the outer tubing.

13. The endoscopic instrument of claim 12, wherein the plurality of segments comprises a plurality of first segments having a rigidity greater than a threshold rigidity and a plurality of second segments having a rigidity less than the threshold rigidity, the plurality of second segments positioned in alternating order with the plurality of first segments.

14. The endoscopic instrument of claim 12, wherein the plurality of segments each comprise an angled surface facing an adjacent segment of the plurality of segments, and the at least one control member comprises a plurality of wires coupled with one or more segments of the plurality of segments such that selectively applying tension to one or more wires of the plurality of wires as the control input controls the orientation of the at least one segment of the plurality of segments in at least two axes.

15. The endoscopic instrument of claim 12, wherein the plurality of segments each comprise a radiused surface facing an adjacent segment of the plurality of segments, and the at least one control member comprises a plurality of wires coupled with one or more segments of the plurality of segments such that selectively applying tension to one or more wires of the plurality of wires as the control input controls the orientation of the at least one segment of the plurality of segments in at least two axes.

16. The endoscopic instrument of claim 12, wherein the plurality of segments each comprise at least one of an electroactive polymer, a shape memory alloy, an electromagnetic coil, or a permanent magnet, and the at least one control member is configured to output at least one of an electrical field or a magnetic field to control a shape of the plurality of segments.

17. The endoscopic instrument of claim 12, wherein the end effector comprises at least one of a snare or a cutting member that extends beyond an outlet of the at least one of the instrument channel of the external sheath.

18. The endoscopic instrument of claim 12, further comprising an outer cutter coupled to a distal end of the articulation assembly, the outer cutter defining a cutting window, wherein the end effector comprises an inner cutter configured to rotate within the outer cutter responsive to actuation of the flexible torque component by a drive assembly coupled to a proximal end of the flexible torque component.

19. A method for controlling an end effector of an endoscopic instrument, comprising:
providing the endoscopic instrument through at least one of an instrument channel of an endoscope or an external sheath coupled to the endoscope, a distal end of the endoscope positioned in proximity to a site within a subject;
determining an orientation at which to articulate the end effector to contact material at the site within the subject;
providing a first control input to at least one control member of the endoscopic instrument based on the orientation to cause an articulation assembly of the endoscopic instrument to move the end effector to the orientation; and
providing a second control input to a flexible torque component coupled to the end effector to cause the end effector to interact with the material responsive to the second control input.

20. The method of claim 19, further comprising actuating a locking assembly to restrict movement of the at least one control member to set the articulation assembly to the orientation.
